(19) United States
(12) Patent Application Publication
(10) Pub. No.: US 2025/0360165 A1
(43) Pub. Date: Nov. 27, 2025

(54) SILICIC ACID FOR IMPROVING MILK PRODUCTION IN DAIRY LIVESTOCK

(71) Applicant: BARLAA B.V., Muiden (NL)

(72) Inventors: Henk Maarten LAANE, Muiden (NL); Cornelis Hendrik Geuvel VAN STEE, Hendrik Ido Ambacht (NL)

(73) Assignee: BARLAA B.V., Muiden (NL)

(21) Appl. No.: 19/107,064

(22) PCT Filed: Sep. 1, 2023
(86) PCT No.: PCT/EP2023/074046

§ 371 Date: Feb. 27, 2025

(30) Foreign Application Priority Data
Sep. 1, 2022 (EP) 22193413.6

(51) Int. Cl. A61K 33/00; A61K 9/00; A61P 43/00

(52) U.S. Cl. CPC A61K 33/00; A61K 9/0053; A61K 9/0095; A61P 43/00

(57) ABSTRACT

The present invention concerns the field of dairy farming, such as farming of cows, buffaloes, camels, yaks, goats, sheep, horses, donkeys, alpacas, mithun, lamas, zebus, etc. In these fields there is still an unmet need for new modalities that can improve milk production, improve milk quality, improve feed efficiency and/or and/or reduce environmental impact. The present invention resides in the finding that this can be realized by using bioavailable forms of silicic acid, typically as an additive that can be fed to the animals via the feed, forage and/or drinking water.

### FIELD OF THE INVENTION

The present invention concerns the field of dairy production. More in particular, the present invention relates to the use of bioavailable silicic acid compounds in dairy livestock farming so as to improve productivity, especially to enhance milk production, improve milk quality, improve feed efficiency, etc. The present invention also relates to dairy livestock farming methods wherein said bioavailable silicic acid compound is used, as well as to specific compositions comprising bioavailable silicic acid compounds, which are specifically adapted for use in dairy livestock farming.

### BACKGROUND OF THE INVENTION

Milk and milk components from dairy livestock are utilized in the preparation of foodstuffs in many different forms. In the last three decades, world milk production has increased by more than 59 percent, from 530 million tonnes in 1988 to 843 million tonnes in 2018. India is the world's largest milk producer, with 22 percent of global production, followed by the United States of America, China, Pakistan and Brazil. Since the 1970s, most of the expansion in milk production has been in South Asia, which is the main driver of milk production growth in the developing world. The countries with the highest milk surpluses are New Zealand, the United States of America, Germany, France, Australia and Ireland. The countries with the highest milk deficits are China, Italy, the Russian Federation, Mexico, Algeria and Indonesia.

In recent decades, developing countries have increased their share in global dairy production. Milk production in Africa is growing more slowly than in other developing regions, because of poverty and—in some countries—adverse climatic conditions. In many regions of the world, growth is mostly the result of an increase in numbers of producing animals rather than a rise in productivity per head. For instance, in countries such as Bangladesh and Nigeria, the average cattle milk yield is ≤500 kg/year. In countries with more developed dairy sectors, such as Iran, Peru and Vietnam, the average cattle milk yield is >1.500 kg/cow/year. In Europe the yields are higher up to 7000-8000 kg/cow/year. Low productivity per head is often the result of poor-quality feed resources, occurrence of diseases, limited access to markets and services (e.g., health, credit and training) and dairy animals' low genetic potential for milk production. Unlike developed countries, many developing countries have hot and/or humid climates that are unfavourable for dairying.

There is recognition of the need to improve the performance of the diary sector in order to meet the growing global demands for dairy products and to lower or minimize its environmental impacts. Improvements in livestock productivity, feed-use efficiency and wise sourcing of feeds are believed to reduce the sector's demand for resources such as land and water, and its environmental impact.

Forages have the greatest effect on feed efficiency. Since they make up a very large component of the slowly digestible part of the diet of lactating cows, they are critical for maintaining a desired level of feed efficiency (FE). They also have a large impact on FE because they are the most variable feed ingredient in terms of digestibility and nutrient composition and they comprise a greater proportion of the ration than any other feedstuff. It has been shown that increased forage digestibility leads to increased FE. Another way in which forage influences FE is through the maintenance of a desirable rumen environment. Acidosis (low rumen pH) can negatively affect FE by decreasing fiber digestibility through changes in the rumen microbial profiles. Adequate physically effective fiber (forage particle size) in the ration will maintain the proper rumen environment by stimulating chewing and ruminating, increasing saliva secretion, and improving buffering capacity of the rumen.

With the focus on feed efficiency and productivity in livestock operations, the production of high-quality forage increasingly tends to compete with food availability for humans. For this reason, feed additives have attracted more and more attention over the past years as a means to enhance feed efficiency and productivity.

For instance, there is evidence that feeding yeast, ionophores, and direct-fed microbials to lactating dairy cows can increase feed efficiency, especially when cows are heat stressed. These additives generally increase FE by positively affecting fiber digestion. RONOZYMER RumiStar™ contains α-amylase which increases the speed of starch degradation in the rumen. It catalyzes starch hydrolysis to oligosaccharides, which are used as an energy source by fibrolytic bacteria. This improves cell-wall degradation, resulting in better fiber digestion. WO2016/128530 concerns combinations of amylase and essential oil mixtures, which are said to specifically improve digestibility of corn diets.

Currently known feed additives have one or more downsides, e.g. in that the gains are usually limited, they are not widely available, are not affordable, especially in the less developed regions of the world, are not universally applicable, etc.

As will be understood there is still an unmet need for new and improved modalities in dairy farming that can improve milk production, milk quality, feed efficiency and growth of the animals and. It is the object of the present invention to satisfy this need.

### SUMMARY OF THE INVENTION

Generally stated, the present invention resides in the finding that the aforesaid objective can be realized by using bioavailable forms of silicic acid. Numerous experiments, some of which are described in the experimental section here below, have shown that the use of certain bioavailable silicic acid compounds in dairy livestock has a remarkable, beneficial impact on milk production, milk quality, feed efficiency and growth of the animals. The remarkable beneficial effects that have been observed in the various trials are probably intertwined and interdependent and the various mechanisms potentially involved have not yet been elucidated in full.

The use of bioavailable forms of silicic acid to treat animals has been described in WO 03/101915. According to WO 03/101915 the treatment of animals (and humans) with products comprising an aqueous solution of boric acid and non-colloidal silicic acid can be used for strengthening connective tissue, bones, skin, nails, arteries, cartilage and joints. As regards the actual treatment of animals, WO 03/101915 only describes a trial in horses, where the main finding was that the treatment resulted in strengthening of the hooves. Based on these results, WO 03/101915 teaches to employ the non-colloidal silicic acid and boron containing solutions for treating animals. WO 03/101915 does not teach any other effects of the treatment on milk production, milk quality, feed efficiency and growth of the animals in dairy farming

To the inventors' best knowledge, they have been the first to show that the administration of bioavailable silicic acid compounds favorably affects productivity, milk quality, feed efficiency and growth of the animals.

Hence, a first aspect of the invention concerns the use of compositions comprising a bioavailable silicic acid compound in dairy farming, especially the farming of dairy livestock, such as cows, cattle, buffaloes, yaks, goats, sheep, alpacas, camels, mithun, lamas, donkeys, etc., typically as an additive for feed and/or drinking water.

A further aspect of the invention concerns a method of dairy farming, especially the farming of dairy livestock, such as cows, cattle, buffaloes, yaks, goats, sheep, alpacas, camels, mithun, lamas, donkeys, etc., said method comprising the step of administering to said animals a composition comprising a bioavailable silicic acid compound.

A further aspect of the invention concerns a method of dairy farming, especially the farming of dairy livestock, such as cows, cattle, buffaloes, yaks, goats, sheep, alpacas, camels, mithun, lamas, donkeys, etc., said method comprising the step of adding a bioavailable silicic acid compound to feed and/or drinking water and feeding it to said.

A further aspect of the invention concerns a non-therapeutic method of treating dairy livestock animals, such as cows, cattle, buffaloes, yaks, goats, sheep, alpacas, camels, mithun, lamas, donkeys, etc., said method comprising the step of administering to said animals a composition comprising a bioavailable silicic acid compound.

A still further aspect of the present invention concerns a composition comprising a bioavailable silicic acid compound for use in the therapeutic or prophylactic treatment of dairy livestock animals, such as cows, cattle, buffaloes, yaks, goats, sheep, alpacas, camels, mithun, lamas, donkeys, etc.

A still further aspects of the present invention concerns a composition comprising a bioavailable silicic acid compound for use in the manufacture of a product for the therapeutic or prophylactic treatment of dairy livestock animals, such as cows, cattle, buffaloes, yaks, goats, sheep, alpacas, camels, mithun, lamas, donkeys, etc.

A still further aspects of the present invention concerns a method of treating a diary livestock animal, such as cows (or cattle), buffaloes, yaks, goats, sheep, alpacas, camels, mithun, lamas, donkeys, etc., by administering to the animal a composition comprising a bioavailable silicic acid compound.

These and other aspects of the invention as well as the preferred embodiments thereof will become apparent to those skilled in the art, based on the following detailed description and examples.

### DETAILED DESCRIPTION OF THE INVENTION

As will be apparent to those skilled in the art, based on the present teachings, the compositions used in accordance with the invention comprise a bioavailable silicic acid compound.

In the context of the present invention, the term ‘silicic acid’ is used to refer to compounds with the basic structure [SiO₂₋ₓ(OH)₂ₓ(H₂O)ₘ]ₙ, wherein x=0 or 1; m=0, 1 or 2; and n≥1. Such compounds thus comprise orthosilicic acid (Si(OH)₄) as the fundamental building block. Si(OH)₄ is a relatively unstable and tends to undergo autocondensation into dimers (2Si(OH)₄↔(HO)₃Si—O—Si(OH)₃+H₂O), trimers ((HO)₃Si—O—Si(OH)₃+Si(OH)₄↔(HO)₃Si—O—Si(OH)₂—O—Si(OH)₃+H₂O), etc., to form oligomers and/or polymers. The formation of small-size particles (non-colloids, sub-colloids and micro-colloids, colloids) is a gradual process. This process eventually results in the formation of a soft gel, which is poorly bioavailable. The formation of colloids and gels is pH dependent. The longest gelling time occurs at pH 2. At lower and more alkaline pH, the time for colloid and finally gel formation decreases (Ralph K. ller. The Chemistry of Silica. Wiley: New York, 1979). The stages from monomer to sol-gel polymerization can be summarized as follows:
1. monomeric orthosilicic acid in acid medium;
2. polymerization of orthosilicic acid, from monomers into dimers, trimers, tetramers, linear or cyclic oligomers up to structures of more than thousand silicic acid monomers;
3. further condensation into linear or randomly branched polymers, which typically take the form of small spherical particles, having a particle size of between 1-10 nm, referred to as ‘subcolloidal’, consisting of several thousands of silicic acid monomers;
4. growth of these particles to a particle size of about 10-100 nm, referred to as colloidal;
5. linking of particles into chains (aggregation);
6. chained into network and extension throughout the liquid (aggregation, pre-gel);
7. thickening into a gel.

The term ‘bioavailable’ as used in the context of the present invention refers to silicic acid provided in a form that may enter into living organisms. Bioavailable forms of silicic acid include, in particular, monomeric silicic acid (also referred to as orthosilicic acid) as well as dimeric silicic acid, which is believed to exist in equilibrium with monomeric silicic acid in aqueous systems.

The term ‘bioavailable silicic acid compound’, is used herein to embrace compounds with the basic structure [SiO₂₋ₓ(OH)₂ₓ(H₂O)ₘ]ₙ that are in a form capable of releasing/liberating monomeric silicic acid (i.e. by depolymerization reactions), e.g. when dispersed in water or an aqueous system. Such bioavailable silicic acid compounds include, in particular, the stage 2 and 3 compounds as defined here above, in addition to monomeric silicic acid (also referred to as orthosilicic acid) and dimeric silicic acid. Hence, in preferred embodiments of the invention, the bioavailable silicic acid compound is selected from the group consisting of monomeric silicic acid (also referred to as orthosilicic acid), dimeric silicic acid, oligomeric silicic acid and polymeric silicic acid in subcolloidal form and combinations thereof.

Preferably, in the compositions employed in accordance with the present invention, at least 50 mol. % of the silicon contained in the composition is in the form of a bioavailable silicic acid compound as defined herein, more preferably at least 60 mol. %, still more preferably at least 70 mol. %, still more preferably at least 75 mol. %, still more preferably at least 80 mol. %, still more preferably at least 85 mol. %, still more preferably at least 90 mol. %, still more preferably at least 95 mol. %, still more preferably at least 97.5 mol. %. It will be understood by those skilled in the art that the term ‘silicon’ is used here as having its (only) conventional meaning, i.e. as denoting the chemical element with the symbol Si. Consequently, when, in the present disclosure a certain mol. % of silicon contained in the composition is recited (as being in the form of a bioavailable silicic acid compound), it denotes the percentage of the total number of Si atoms contained in the composition (that is in the form of a bioavailable silicic acid compound).

The composition employed in accordance with the present invention, preferably comprise subcolloidal silicic acid, i.e. silicic acid that is mainly in stages 2 and 3 as defined here above. Solutions comprising such subcolloidal particles passes through a 0.1 micron filter. The invention is not directed to uses of and/or methods employing silicic acid in colloidal form or in sol form. Although minor amounts of these species may be present in the compositions of the invention, the compositions of the invention substantially comprises non-colloidal silicic acid (i.e. compounds with the basic structure [SiO₂₋ₓ(OH)₂ₓ(H₂O)ₘ]ₙ that are mainly in stage 2 and stage 3, as described above).

In particularly preferred embodiments of the invention, the bioavailable silicic acid compound is subcolloidal silicic acid, more preferably silicic acid in the form of subcolloidal particles having a size of 10 nm or less, more preferably 8 nm or less, still more preferably 6 nm or less, still more preferably 5 nm or less, most preferably 4 nm or less. Furthermore, in particularly preferred embodiments of the invention, the bioavailable silicic acid compound is subcolloidal silicic acid, more preferably silicic acid in the form of subcolloidal particles having a size within the range of 1-10 nm, more preferably within the range of 1.5-8 nm, still more preferably 2-6 nm, still more preferably 3-5 nm, most preferably 3.5-4 nm. Particle size determinations can be made using ²⁹Si NMR spectroscopy, TEM and/or SEM. In preferred embodiments of the invention, at least 50% of the silicic acid containing particles in the compositions have a particle diameter within the aforementioned size ranges, more preferably at least 60%, still more preferably at least 70%, still more preferably at least 75%, still more preferably at least 80%, still more preferably at least 85%, still more preferably at least 90%, most preferably at least 95%. It will be understood by those skilled in the art, that the aforementioned percentages express the number of particles that comply with the indicated particle size characteristic, relative to the total number of particles.

The composition employed in accordance with the present invention, typically have the form of aqueous dispersions or solutions of the bioavailable silicic acid compound at adequate concentrations that can be added to the drinking water and/or feed or forage fed to the dairy livestock animals in a practical manner. Although the invention is not particularly limited in this regard, preferred embodiments are envisaged wherein the composition as employed comprises bioavailable silicic acid compounds at a level of at least 0.01 ppm, at least 0.05 ppm, at least 0.1 ppm, at least 0.5 ppm, at least 1 ppm, at least 5 ppm, or at least 10 ppm. Furthermore, preferred embodiments are envisaged wherein the composition as employed comprises bioavailable silicic compounds at a level below 5000 ppm, e.g. below 1000 ppm, below 500 ppm, below 100 ppm, below 50 ppm or below 10 ppm.

The afore defined aqueous solutions or dispersions may typically be produced from a highly concentrated aqueous product or a product in dry solid form, i.e. by diluting/mixing such a product with an adequate quantity of water or with an adequate quantity of feed just before actual use. It is known that aggregation of subcolloidal silicic acid particles (into the forms of stage 4 or higher) may occur over time, especially in the case of highly concentrated products, resulting in opalescence, turbidity, light reflection, colloid and gel formation and thus loss of bioactivity upon storage. Hence, such products in concentrated or dry solid form may contain additives effective in preventing formation of colloidal or macrocolloidal silicic acid particles. International patent application no. WO 2003/101915 and international patent application no. WO 2011/071379, both incorporated herein by reference, describe various techniques to stabilize concentrated products comprising bioavailable silicic acid compounds. Hence, the compositions employed in accordance with the present invention may contain additives, such as those taught by WO 2003/101915 and WO 2011/071379. As will be understood by those skilled in the art though, based on the present teachings, the presence of such additives in the compositions employed per se is not critical or essential for attaining the beneficial effects in dairy farming described herein; what counts is that the composition employed contains bioavailable silicic acid compounds, irrespective of how it is made and provided and/or what measures may have been taken to stabilize it during (prolonged) storage. Notwithstanding the former, from a practical standpoint, the compositions taught by WO 2003/101915 and WO 2011/071379 may have advantages for the purposes of the present invention. Hence, in certain preferred embodiments of the invention, the composition comprises an acidified aqueous solution of (1) subcolloidal silicic acid in combination with (2) boric acid and/or (3) a water absorbing additive. In preferred embodiments, said water absorbing additive comprises a humectant selected from the group consisting of a polysorbate, a vegetable gum, a substituted cellulose, a polyglycerol ester of a fatty acid, a polyethylene glycol, a polydextrose, a propylene glycol, a propylene glycol alginate, a polyoxyethylene fatty acid ester, a pectine or amidated pectine, a sucrose ester of a fatty acid, an acetylated or hydroxypropyl starch, a starch phosphate, urea, sorbitol, malitol, (pro-)vitamins, and a mixture of two or more of such humectants. Preferably, the water absorbing additive concentration is at least 10 wt. % of the composition, based on dry solids weight, such as at least 25 wt. %, at least 40 wt. % or at least 50 wt. %. The water absorbing additive concentration is typically less than 75 wt. % of the composition, based on dry solids weight, e.g. less than 70 wt. %, less than 65 wt. % or less than 60 wt. %. In embodiments where the bioavailable silicic acid compound is combined with boric acid, preferably the molar Si/B ratio is in the range of 0.1-1000, more preferably 0.5-500, 1-400 or 1.5-300. In preferred embodiments, the composition is filterable through a 0.1 micron filter. In preferred embodiments, the composition is filterable through a 20,000 Mw (Da) filter.

Exemplary composition that is particularly suited for use in accordance with the present invention is the product commercialized by RexilAgro b.v. (the Netherlands) under the trade names AB Silicow®, AB Siliyak®, AB Silichicken@, AB Silifish®, OSAB®, OSAB3®, OSABPLUS® and OSABSIPLUS®.

Compositions comprising bio-available silicic acid that are also particularly suited and preferred for the methods and uses of the present invention, include those defined by European Patent application no. EP22188329.1, the contents of which are incorporated herein by reference.

Other types of bio-available silicic acid are known in the art and/or are commercially available as well, the suitability of which, for the purposes of the present invention, will be dependent on the specific technology and chemicals used to stabilize the bioavailable silicic acid. It is within the routine capabilities of the person skilled in the art, to make such determinations, taking into account the present teachings.

In preferred embodiments of the invention, the composition may further comprise one or more additional nutrients selected from the group consisting of zinc, manganese, copper, molybdenum, selenium, humic acid, a fulvic acid, amino acids, etc. In further preferred embodiments of the invention, the composition may comprise one or more additional feed additives conventionally used in dairy livestock farming.

An aspect of the present invention provides the compositions adapted for the uses and methods of the present invention per se, e.g. any of the compositions as defined here above, including the concentrated and dry solid form products that need to be mixed/diluted with water prior to actual use. In a preferred embodiment of the invention, a product is provided in the form of a container comprising a composition as defined here above, which may be a concentrated product or dry solid form product that needs to be mixed/diluted with water prior to actual use, wherein said container is provided with instructions printed on the container and/or instructions printed on a label provided with the container, to use the composition for the purposes and/or in the manners as defined herein.

As will be understood by those skilled in the art, based on the present teachings, the methods and uses of the present invention entail the addition of the composition comprising bioavailable silicic acid compounds, preferably a composition as defined herein before, to the feed compositions and/or drinking water fed to the animals, so as to attain one or more of the beneficial effects mentioned herein (such as increased growth, increased feed conversion ratio, increased milk production, improved milk quality, etc.). In accordance with the invention, the compositions comprising bioavailable silicic acid compounds can be added to the feed and/or drinking water separately. Embodiments are also envisaged though, wherein the composition is mixed or blended with other products added to the feed and/or drinking water in typical dairy farming operations, such as other feed additives, vitamins, minerals, etc.

For optimal results the uses and methods entail the addition of the composition comprising bioavailable silicic acid compounds to the drinking water in quantities resulting in level of at least 0.1 ppm of bioavailable silicic acid compound in the water, preferably at least 0.5 ppm, at least 1 ppm, at least 2.5 ppm, at least 5 ppm or at least 10 ppm, e.g. about 25 ppm of bioavailable silicic acid compound in the drinking water. Furthermore, in preferred embodiments of the invention, the uses and methods entail the addition of the composition to the drinking water in quantities resulting in a level of less than 1000 ppm of bioavailable silicic acid compound in the water, preferably less than 750 ppm, less than 500 ppm, less than 250 ppm, less than 100 ppm or less than 50 ppm.

In other preferred embodiments of the invention, the uses and methods entail the addition of the composition comprising bioavailable silicic acid compounds to the feed, i.e. the/a standard feed or forage composition conventionally used in the farming of the species in suit, in quantities resulting in level of at least 0.001 ppm of bioavailable silicic acid compound in the feed, preferably at least 0.005 ppm, at least 0.01 ppm, at least 0.025 ppm, at least 0.05 ppm or at least 0.10 ppm, e.g. about 0.25 ppm. Furthermore, in preferred embodiments of the invention, the uses and methods entail the addition of the composition to the feed in quantities resulting in a level of less than 10 ppm of bioavailable silicic acid compound in the feed, preferably less than 7.5 ppm, less than 5 ppm, less than 2.5 ppm, less than 1 ppm or less than 0.5 ppm.

In other preferred embodiments of the invention, the uses and methods entail administration of bioavailable silicic acid compounds to the animals, in dosages of bioavailable silicic acid compound of at least 0.001 mg/kg, preferably at least 0.005 mg/kg, at least 0.01 mg/kg, at least 0.025 mg/kg, at least 0.05 mg/kg or at least 0.1. Furthermore, in preferred embodiments of the invention, the uses and methods entail administration of bioavailable silicic acid compounds to the animals, in dosages of bioavailable silicic acid compound of less than 5 mg/kg, preferably less than 2.5 mg/kg, less than 1 mg/kg, less than 0.5 mg/kg, less than 0.25 mg/kg or less than 0.1 mg/kg.

With a view to optimal results, it is preferred that feed enriched with the bioavailable silicic acid compounds is given to the animal repeatedly, e.g. the uses and methods entail the repeated administration of bioavailable silicic acid compounds in accordance with the invention, e.g. at least once every 10 days, at least once every 7 days, at least once every 5 days, at least once every 3 days, at least once every other day or at least once every day, such as up to two times a day or three times a day. In particularly preferred embodiments of the invention, the animals to be treated receive bioavailable silicic acid compounds in accordance with the invention every day, preferably once every day, twice every day, three times every day or four times every day.

In preferred embodiments of the invention the administration of bioavailable silicic acid compounds according to the regimens defined above, is continued for a period of at least 1 week, at least two weeks, at least three week, at least four weeks, at least one month, at least two months, at least three months or at least four months. In preferred embodiments of the invention addition of the composition comprising bioavailable silicic acid compounds to the water and/or to the feed is according to the regimens defined above, is carried out substantially or entirely throughout the life span or life cycle of the animals.

In preferred embodiments of the invention, the animal species is selected from the group consisting of dairy livestock animals in its broadest sense. As is known by those skilled in the art, differences may exist between different regions of the world, with regard to the animal species that are most typically used for diary production and the present invention is not particularly limited with regard to the exact type of livestock animals that may be treated. In certain embodiments of the invention, the livestock animal to be treated by the administration of the bioavailable silicic acid compound is selected from one of the following families: Bovidae Camelidae and Equidae. In certain embodiments of the invention, the livestock animal to be treated by the administration of the bioavailable silicic acid compound is selected from one of the following genera: In certain embodiments of the invention, the livestock animal to be treated by the administration of the bioavailable silicic acid compound is selected from the group consisting of cows, buffaloes, camels, yaks, goats, sheep, horses, donkeys, alpacas, mithun, lamas and zebus, most preferably from the group consisting of cows, goats and sheep.

In certain preferred embodiments of the invention, the animal species is not a horse, more preferably the animal species is not a species from the genus equus, more preferably it is not a species from the family of Equidae.

As will be understood by those skilled in the art, the animals to be treated are of the female gender, have reached adulthood and are in a lactation phase. In preferred embodiments of the invention, the animals to be treated are in the early lactation phase, the mid-lactation phase and/or the late lactation phase, most preferably in the mid-lactation phase and/or the late lactation phase, most preferably in the later lactation phase. In preferred embodiments of the invention, the animals to be treated are bovines, which are in the 1^{st} to 12^{th} month of lactation, such as in the 2^{th} to 12^{th} month of lactation, the 3^{th} to 12^{th} month of lactation, the 4^{th} to 12^{th} month of lactation, the 1^{st} to 10^{th} month of lactation, the 2^{th} to 10^{th} month of lactation, the 3^{th} to 10^{th} month of lactation, the 4^{th} to 10^{th} month of lactation, the 1^{st} to 8^{th} month of lactation, the 2^{th} to 8^{th} month of lactation, the 3^{th} to 8^{th} month of lactation, the 4^{th} to 8^{th} month of lactation.

In preferred embodiments of the invention, the animals to be treated are in a good health, are in a good condition, do not suffer from ill-health, do not suffer from a disease, do not suffer from a pathology and/or do not suffer from a health condition. In preferred embodiments of the invention the animals to be treated are not at a particular risk and/or at an increased risk of suffering from a disease, pathology or health condition.

As already stated herein before, the methods and uses of the invention result in and/or are aimed at the attainment of one or more beneficial effects in relation to milk production, milk quality, feed efficiency and animal growth. In the methods and uses of the invention, as defined herein, the composition comprising the bioavailable silicic acid compound may be regarded a feed additive, such as a technological feed additive, a nutritional feed additive or a zootechnical additive. These terms and their meaning, in the context of the present invention, are well known and understood by those skilled in the art.

In one embodiment of the invention, the methods and uses result in and/or are aimed at increasing the overall milk yield. In particularly preferred embodiments of the invention, yield is increased by at least 5%, typically on a weight basis, compared to yield attained under the same conditions but without the silicic acid treatment. In particularly preferred embodiment said yield is increased by at least 7.5%, at least 10%, at least 12.5%, at least 15%, at least 17.5% or at least 20%.

In particularly preferred embodiments of the invention the animal is a bovine or cow and the methods of the invention result in and/or are aimed at increasing the yield by at least 0.5 kg per day per animal, compared to yield attained under the same conditions but without the silicic acid treatment. In particularly preferred embodiment said yield is increased by at least 0.75 kg per day per animal at least 1 kg per day per animal at least 1.25 kg per day per animal, at least 1.5 kg per day per animal, at least 1.75 kg per day per animal, at least 2 kg per day per animal, at least 2.25 kg per day per animal or at least 2.5 kg per day per animal.

In particularly preferred embodiments of the invention the animal is a bovine or cow and the methods of the invention result in and/or are aimed at increasing the yield by at least 0.5 liter per day per animal, compared to yield attained under the same conditions but without the silicic acid treatment. In particularly preferred embodiment said yield is increased by at least 0.75 liter per day per animal at least 1 liter per day per animal at least 1.25 liter per day per animal, at least 1.5 liter per day per animal, at least 1.75 liter per day per animal, at least 2 liter per day per animal, at least 2.25 liter per day per animal or at least 2.5 liter per day per animal.

In particularly preferred embodiments of the invention the animal is a bovine or cow and the methods of the invention result in and/or are aimed at increasing and/or maintaining the yield at a level of at least 12.5 liter per day per animal, preferably at least 13 liter per day per animal, at least 13.5 liter per day per animal, at least 14 liter per day per animal, at least 14.5 liter per day per animal or at least 15 liter per day per animal.

In particularly preferred embodiments of the invention, the animal is a bovine or cow and the methods of the invention result in and/or are aimed at increasing, yield expressed as 3.5% FCM milk, by at least 0.5 kg per day per animal, compared to yield attained under the same conditions but without the silicic acid treatment. As used herein, the term “FCM milk” refers to Fat Corrected Milk, which is a measure that is used in the art to standardize milk production and enable meaningful comparisons, by combining actual milk and fat yields into one value that repreents the volume of milk at the specified fat percentage. For this purpose various conversions can typically be chosen. The standard formula for FCM 3.5% fat is as follows:

$$FCM ( 3.5 \% ) = 0.35 M + 18.57 F$$

where M=quantity of milk in kg, F=amount of fat in kg ‘M’ quantity of milk. In particularly preferred embodiment said yield is increased by at least 0.75 kg per day per animal at least 1 kg per day per animal at least 1.25 kg per day per animal, at least 1.5 kg per day per animal, at least 1.75 kg per day per animal, at least 2 kg per day per animal, at least 2.25 kg per day per animal or at least 2.5 kg per day per animal.

In particularly preferred embodiments of the invention the animal is a bovine or cow and the methods of the invention result in and/or are aimed at increasing and/or maintaining the yield, expressed as 3.5% FCM milk, to/at a level of at least 33.5 kg per day per animal, preferably at least 34 kg per day per animal, at least 34.5 kg per day per animal at least 35 kg per day per animal, at least 35.5 kg per day per animal or at least 36 kg per day per animal.

In one embodiment of the invention, the methods and uses result in and/or are aimed at improving feed utilization. In one embodiment of the invention, the methods and uses result in and/or are aimed at improving the feed conversion rate, referred in the art as FCR, which is a measure of an animal's efficiency in converting feed mass into increases of the desired output. For food-producing animals, the output is the mass gained by the animal, while for dairy animals, the output is milk. Specifically, unless otherwise explicitly stated in the disclosure, FCR is calculated herein as mass of feed per mass of milk, all over a specified period. Improvement in feed utilization means reduction of the FCR value, compared to the FCR attained under the same conditions but without the silicic acid treatment. In particularly preferred embodiment, improvements in feed utilization are reflected by an FCR decrease of at least 2.5%, preferably at least 5%, at least 7.5%, at least 10%, at least 12.5%, or at least 15%.

In one embodiment of the invention, the methods and uses result in and/or are aimed at improve the milk quality.

In one embodiment of the invention, the methods and uses result in and/or are aimed at increasing the fat content of the milk, typically with at least 0.5 wt. %. As used herein, the term ‘fat content of the milk’ refers to the amount of fat relative to the total weight of the milk. In particularly preferred embodiment the fat content is increased with at least 0.25 wt. %, 0.5 wt. %, at least 0.75 wt. %, at least 1 wt. % or at least 1.25 wt. %, compared to the fat level attained under the same conditions but without the silicic acid treatment. In another particularly preferred embodiment the fat level of the milk is increased to a level, and maintained at a level, of above 4.1 wt. %, preferably above 4.25 wt. %, above 4.5 wt. %, above 4.75 wt. %, above 5 wt. %, or above 5.25 wt. %.

In one embodiment of the invention, the methods and uses result in and/or are aimed at increasing the protein content of the milk, typically with at least 0.25 wt. %. As used herein, the term ‘protein content of the milk’ refers to the amount of protein relative to the total weight of the milk. In particularly preferred embodiment the fat content is increased by at least 0.30 wt. %, 0.35 wt. %, at least 0.40 wt. %, at least 0.45 wt. % or at least 0.50 wt. %, compared to the protein level attained under the same conditions but without the silicic acid treatment. In another particularly preferred embodiment the protein level of the milk is increased to a level, and maintained at a level, of above 3.35 wt. %, preferably above 3.4 wt. %, above 3.5 wt. %, above 3.6 wt. %, above 3.7 wt. %, or above 3.8 wt. %.

In one embodiment of the invention, the methods and uses result in and/or are aimed at reducing the ecological impact of dairy farming.

In particularly preferred embodiments of the invention, the methods and uses do not do not have and/or are not intended to have any effects on the health of the dairy livestock animals, such as the curing of a pathology or health condition, preventing a pathology or health condition and/or alleviating one or more symptoms of a pathology or health condition. In particularly preferred embodiments of the invention, the methods and uses are non-therapeutic, e.g. non-curative and non-prophylactic. In a particularly preferred embodiment of the invention the methods and uses do not affect and/or are not aimed at affecting the immune system, such as strengthening the immune system. In a particularly preferred embodiment of the invention the methods and uses do not result in and/or are not aimed at strengthening connective tissue, bones, skin, nails, arteries, cartilage and joints and/or at the prevention or curing of any disease or condition associated with (a deficiency in) the strength of connective tissue, bone tissue, skin tissue, nail, arteries, cartilage and/or joints. In a particularly preferred embodiment of the invention the methods and uses involve the treatment of healthy animals, animals that do not suffer and/or are not known to suffer from any pathology or health condition.

Although, the treatments in accordance with the present invention result in the productivity improvements described herein when applied to healthy animals and irrespective of the ailment of any pathology or health condition, positive health effects in animals suffering from mastitis have been reported. Hence in further embodiments of the present invention, the uses and methods of the treatment result in and/or are aimed at alleviating, curing and/or preventing mastitis in dairy livestock animals. Hence, further aspects of the invention relate to a method of treating or preventing mastitis in a dairy livestock animal, said method comprising the administration of a composition comprising a bioavailable silicic acid compound, as defined herein; to the use of a composition comprising a bioavailable silicic acid compound, as defined herein, in the manufacture of a medicament for use in the treatment and/or prevention of mastitis in a diary livestock animal; and to a composition comprising a bioavailable silicic acid compound, as defined herein, for use in a method treating and/or preventing mastitis in a diary livestock animal. Such methods of treating and/or preventing mastitis in a diary livestock animal involve the administration of the bioavailable silicic acid compound and compositions comprising, in accordance with the present disclosure, following the treatment protocols and regimens as defined herein before.

In a certain preferred embodiment of the invention the methods and uses do not affect and/or are not aimed at treating and/or preventing mastitis. Furthermore, in certain preferred embodiments of the invention the animals to be treated do not suffer from mastitis.

Unless otherwise defined, all terms used in disclosing the invention, including technical and scientific terms, have the meaning as commonly understood by one of ordinary skill in the art to which this invention belongs.

“A”, “an”, and “the” as used herein refer to both singular and plural forms unless the context clearly dictates otherwise. By way of example, “a compartment” refers to one or more than one compartment.

“About” as used herein referring to a measurable value such as a parameter, an amount, a temporal duration, and the like, is meant to encompass variations of +/−10% or less, more preferably +/−5% or less, even more preferably +/−1% or less of and from the specified value, in so far such variations are appropriate to perform in the disclosed invention. However, it is to be understood that the value to which the modifier “about” refers is itself also specifically disclosed.

“Comprise”, “comprising”, “comprises” and “comprised of” as used herein are synonymous with “include”, “including”, “includes” or “contain”, “containing”, “contains” and are inclusive or open-ended terms that specify the presence of what follows, e.g. a component, and do not exclude or preclude the presence of additional, non-recited components, features, element, members, steps, known in the art or disclosed therein.

The recitation of numerical ranges by endpoints includes all numbers and fractions subsumed within that range, as well as the recited endpoints. The skilled person will appreciate that the present invention can incorporate any number of the specific features described above.

Throughout this text, the use of terms in brackets, usually means that the term within brackets specifies a possible option or a possible meaning and should thus not be considered limiting.

Advantages of the invention will become apparent from the following examples, which are given below as mere illustrations, and are non-limitative.

### EXPERIMENTAL

### Example 1—Trial with Silicic Acid on Cows

A trial was done to study the effects of silicic acid as a feed supplement on milk production and overall efficiency in healthy dairy cows.

### Materials and Methods

On a dairy farm, VB Veteran College, NAU, Navsari, South, 4 groups of 10 dairy cows were selected for this study.

Groups were mixed for age, lactation stage, gestation, production level and assigned to control
Group 1:5 ml silicic acid containing additive/day
Group 2:10 ml silicic acid containing additive/day
Group 3:15 ml silicic acid containing additive/day
Group 4: Control (no silicic acid).

Silicic acid containing additive/day groups (1-3): the treatment groups (1-3) were fed Silicic acid containing additive containing stabilized silicic acid 0.8% (Siliyak® ex ReXil Agro BV, the Netherlands).

Milk-, fat- and protein production levels were recorded every 14 days for a period of two months. After the first month, groups were shifted to another Silicic acid level.

### Results and Discussion

The milk production data showed improvement for milk, % fat and FPCM (fat and protein corrected milk) for all feed levels compared to the control group. There is only a slight decrease for % protein for all Silicic acid feed levels. A dosage of 5 ml Silicic acid containing additive/kg of feed is most effective for increased milk production, whereas 10 ml silicic acid containing additive/kg feed is more effective for increasing fat and FPCM.

15 ml silicic acid containing additive/Kg of feed is suboptimal as the higher dose responds with lower production compared to 5 ml. and 10 ml Silicic acid containing additive/kg feed groups.

### Conclusions

The results show that silicic acid has favourable effects on milk production traits for dairy cattle. The silicic acid containing additive offers organic livestock producers a multi nutrient liquid feed supplement with the potential to prevent disease and increase feed conversion at low cost with the further benefit of being able to reduce or increase butterfat levels with varying levels of Silicic acid containing additive in the feed.

### Example 2—Effects of Silicic Acid on the Milk Production of Cows (Cattle) and Goat

A further study was done with the objective of evaluating the effect of silicic acid on the milk production including the protein and milk content of healthy dairy cows and healthy goat.

### Materials and Methods

In several studies the effects of the application of silicic acid containing additive were studied in dairy cows. The silicic acid containing additive was added to the drinking water.

On a (organic) dairy farm in Limzar Farmers' co-operative society Navsari, South Gujrat, 4 groups of 10 dairy cows were selected for this study and compared to control. The cows were fed with a nutrition program to fulfil an optimal diet. The treatment groups (G1-G4) were initially fed an additive containing 0.8% stabilized silicic acid (OSAB® ex ReXil Agro BV, the Netherlands).

The cows were selected on criteria as breed (pedigree), health status, age, lactation stage and production level. Next the cows were divided in 4 groups and a control group (no application of silicic acid). Silicic acid was added to the drinking water in accordance with the following regime:

TABLE 1
treatment regimens

| | |
|---|---|
| C | control, |
| G1 | 2 ml silicic acid containing additive/L |
| | in the drinking water: 2 times/day, |
| G2 | 2 ml/L silicic acid containing additive/L |
| | in the drinking water: 3 times/day |
| G3 | 4 ml/L silicic acid containing additive/L |
| | in the drinking water: 2 times/day |
| G4 | 4 ml/L silicic acid containing additive/L |
| | in the drinking water: 3 times/day. |

Due to (COVID-19 related) supply issues in the course of the trial, the facility temporarily switched to another, locally sourced, stabilized silicic acid containing composition (which was administered at the equivalent dose).

The milk production, the fat and protein levels were recorded every 10 days for a period of 4 months. After the first month, the groups were shifted to another silicic acid level. And the results were recorded.

### Results

The milk production data showed improvement for milk quantity, percentage fat and FPCM (Fat and Protein Corrected Milk) for all feed levels compared to the control group. Especially the group G2 with an application of silicic acid of 2 ml/L, 3 times/day showed the largest increase with 20.2% (=2066 liter/year for G2 being an increase of milk production of 206 L/cow/year.

Also the fat component increased from 36.6 (control) to 42 g/kg in group G2 (+15.7%). The increase of protein component was 2% in groups G1, G3 and G4 and 7% in group G2. Overall Was shown that the 2 times applications are less effective compared to the 3 times applications, but significant more effective compared to control.

TABLE 2
Performance of 5 groups of dairy cows fed with
several levels and frequencies of Silicic acid.

| Specimen | Control | G1 | G2 | G3 | G4 |
|---|---|---|---|---|---|
| Milk yield kg/day | 28 | 29.68 | 33.66 | 30.90 | 29.93 |
| 3.5% FCM milk | 32.80 | 33.85 | 36.45 | 33.92 | 33.98 |
| Fat g/kg | 36.6 | 38.8 | 42.0 | 38.94 | 39.9 |
| Protein g/kg | 30.5 | 31.01 | 32.7 | 31.05 | 31.08 |

TABLE 3A
Intake and digestibility in dairy cows
fed different doses of Silicic acid

| Specimen | Control | G1 | G2 | G3 | G4 |
|---|---|---|---|---|---|
| Dry matter | 20.50 | 20.81 | 22.64 | 20.83 | 20.91 |
| Crude protein | 3.47 | 3.51 | 3.94 | 3.53 | 3.49 |
| Ether extract | 1.03 | 1.05 | 1.09 | 1.07 | 1.06 |
| NDF | 0.5811 | 0.5819 | 0.5864 | 0.5821 | 0.5823 |

TABLE 3B
Intake and digestibility in dairy cows
fed different doses of Silicic acid

| Specimen | Control | G1 | G2 | G3 | G4 |
|---|---|---|---|---|---|
| Dry matter | 0.6689 | 0.6692 | 0.6780 | 0.6695 | 0.6694 |
| Crude protein | 0.6764 | 0.6773 | 0.6824 | 0.6779 | 0.6784 |
| Ether extract | 0.7818 | 0.7822 | 0.7934 | 0.7876 | 0.7877 |
| NDF | 0.5817 | 0.5822 | 0.5864 | 0.5824 | 0.5820 |

The temporary switch to a different kind of silicic acid containing composition in the course of the trial did not result in any noticeable change in the performance parameters in any of the G1-G4 treatment groups.

### Conclusion

The results show that stabilized silicic acid has favorable effects on milk production traits for dairy cattle. The product was shown to be effective in increasing milk production and percentage fat, dependent on concentration and frequency of application. Moreover, The silicic acid containing additive has the potential to increase feed conversion at low cost.

### Example 3—Use of a Silicic Acid Containing Feed Additive in Dairy Cows, Navsari University, Gujarat, India, 2021

In early trials it was established that an animal feed supplement containing bio-available silicic acid increases the nutrient resorption and stimulates the milk production. Based on the initial results a follow-up trial is done in healthy cows.

### Materials and Methods

On an organic dairy farm in Limzar, Farmers' co-operative Society, Navsari, South Gujrat, 4 groups of 10 dairy cows each were selected for this study. The groups were mixed for age, lactation stage, gestation, production level and assigned to respectively a. Control and application of The silicic acid containing additive in different doses, as shown in protocol underneath.

The treatment groups were fed a silicic acid containing additive having a concentration of 0.8% silicic acid (in the undiluted additive; OSAB® ex ReXil Agro BV, the Netherlands).

The milk-, fat- and protein production levels were recorded every 10 days for a period of 6 months. After the first month, groups were shifted to another silicic acid regimen.

50 pregnant Sahiwal cows were selected from the Farmers co-operatives and randomly divided into five groups of ten cows per group. The first group of 10 cows were kept as control; the other six groups were supplemented with the silicic acid containing additive according to dose protocol.

TABLE 4
treatment regimens (amount of silicic acid containing
additive per L of drinking water)

| | |
|---|---|
| G1 | Control, |
| G2 | 2 ml silicic acid containing additive/L - 1 time/day |
| G3 | 2 ml silicic acid containing additive/L - 2 times/day |
| G4 | 3 ml silicic acid containing additive/L - 2 times/day |
| G5 | 3 ml silicic acid containing additive/L - 3 times/day |
| G6 | 4 ml silicic acid containing additive/L - 2 times/day |
| G7 | 4 ml silicic acid containing additive/L - 3 times/day |

Due to (COVID-19 related) supply issues in the course of the trial, the facility temporarily switched to another, locally sourced, stabilized silicic acid containing composition (which was administered at the equivalent dose).

Feed intake after weighing feed refusals was recorded, and DM intake was calculated daily. After calving, the milk yield of the experimental animals was recorded daily up to 120 days of lactation. Milk samples were collected fortnightly from all cows for the analysis of milk composition, and the cows were checked on (sub) clinical mastitis.

Milking of cows was done by milking machine thrice daily at 4:30 AM, 12:00 PM and 7:00 PM. The individual milk yields were recorded in kg at each milking. Milk composition, i.e., fat, protein, lactose and SNF were determined fortnightly by using Lactostar-automatic milk analyzer (Funke Gerber, Model No. 3510-055007).

About 100 ml of milk sample from each individual animal after each milking was collected in a properly cleaned milk sample bottle and pre-warmed at 39-40° C. before analysis.

### Results

TABLE 5
Milk production performance of experimental cows/day/liter

| Specimen | Average Milk yield/day- Lit | Increase in % |
|---|---|---|
| G1 | 12.4 | — |
| G2 | 12.87 | +3.8 |
| G3 | 13.92 | +12.3 |
| G4 | 15.14 | +22 |
| G5 | 15.48 | +24.8 |
| G6 | 14.34 | +15.6 |
| G7 | 14.6 | +17.7 |

TABLE 6
Milk Quality parameters

| Specimen | G1 | G2 | G3 | G4 | G5 | G6 | G7 |
|---|---|---|---|---|---|---|---|
| Average. Milk | 4.01 | 4.1 | 4.15 | 5.34 | 5.30 | 4.10 | 4.12 |
| Fat % | | | | | | | |
| Total solid | 12.03 | 12.50 | 13.03 | 15.68 | 15.72 | 13.25 | 13.3 |
| Solid not fat % | 9.02 | 9.05 | 9.04 | 9.63 | 9.45 | 9.04 | 9.04 |
| Protein % | 3.30 | 3.34 | 3.51 | 3.85 | 4.10 | 3.35 | 3.4 |
| Lactose % | 4.34 | 4.45 | 4.47 | 4.68 | 4.70 | 4.46 | 4.45 |
| Ash % | 0.62 | 0.63 | 0.64 | 0.65 | 0.66 | 0.62 | 0.62 |
| Water % | 86.27 | 86.29 | 86.32 | 86.49 | 86.52 | 86.30 | 86.31 |
| SNF | 8.56 | 8.57 | 8.60 | 8.65 | 8.68 | 8.57 | 8.6 |

The temporary switch to a different kind of silicic acid containing composition in the course of the trial did not result in any noticeable change in the performance parameters in any of the treatment groups.

### Conclusions

The application of the silicic acid containing additive increased the milk production (see Table 1) as well as the milk quality parameters (Table 2).

The best results are achieved with the application of the silicic acid containing additive 2 to 3 times a day (trial arm G4 and G5).

1. Use of a composition comprising a bioavailable silicic acid compound as a feed additive in dairy livestock farming, wherein the bioavailable silicic acid compound is selected from the group consisting of monomeric silicic acid, dimeric silicic acid, oligomeric silicic acid, polymeric silicic acid in subcolloidal form and combinations thereof.

2. Use according to claim 1, wherein at least 90 mol. % of the silicon contained in the composition is in the form of a bioavailable silicic acid compound.

3. Use according to claim 1 or 2, wherein the dairy livestock animals to be treated have reached adulthood and are in a lactation phase.

4. Use according to claim 3, wherein the bioavailable silicic acid compound is in the form of subcolloidal particles having a size of below 10 nm, as determined by ²⁹Si NMR spectroscopy.

5. Use according to claim 4, wherein at least 50% of the silicic acid containing particles has a size within the range of 1-10 nm.

6. Use according to any one of the preceding claims, wherein the composition comprises an acidified aqueous solution or dispersion of bioavailable silicic acid compounds in the form of subcolloidal particles, preferably in combination with boric acid and/or a water absorbing additive.

7. Use according to any one of the preceding claims, wherein the use has the objective of and/or results in one or more of:
increasing milk yield;
improving the feed conversion rate;
improving the milk quality;
increasing the milk fat content;
increasing the milk protein content; and
reducing the ecological impact of dairy livestock farming.

8. Use according to any one of the preceding claims, wherein the use has the objective of and/or results in one or more of:
increasing milk yield;
improving the feed conversion rate;
increasing the milk fat content; and
increasing the milk protein content.

9. Use according to any one of the preceding claims in the farming of a species selected from the group consisting of cows, buffaloes, camels, yaks, goats, sheep, horses, donkeys, alpacas, mithun, lamas and zebus, preferably from the group consisting of cows, goats and sheep.

10. Use according to any one of the preceding claims, wherein the use comprises the addition of the composition comprising bioavailable silicic acid compounds to the drinking water, feed and/or forage fed to the dairy livestock animals.

11. Method of farming dairy livestock animals, comprising the step of adding a composition comprising a bioavailable silicic acid compound to the drinking water, feed and/or forage fed to the dairy livestock animals.

12. Method according to claim 11, wherein the bioavailable silicic acid compound is in the form of subcolloidal particles having a size in the range of 1-10 nm.

13. Method according to claim 11 or 12, wherein at least 90 mol. % of the silicon contained in the composition is in the form of a bioavailable silicic acid compound.

14. Product in the form of a container comprising a composition comprising a bioavailable silicic acid compound, wherein said container is provided with instructions printed on the container and/or instructions printed on a label provided with the container, to use the composition for the purposes defined in any one of claims 1-13.
